(19) United States
(12) Patent Application Publication
(10) Pub. No.: US 2019/0321395 A1
(43) Pub. Date: Oct. 24, 2019

(54) TREATMENT OF PSORIASIS, SEBORRHEIC DERMATITIS, AND ECZEMA OF THE HEAD AND NECK

(71) Applicant: Oread Therapeutics, Lawrence, KS (US)

(72) Inventor: Grant Ghahramani, Lawrence, KS (US)

(21) Appl. No.: 16/390,282

(22) Filed: Apr. 22, 2019

Related U.S. Application Data
(60) Provisional application No. 62/660,513, filed on Apr. 20, 2018.

(51) Int. Cl. A61K 33/04; A61K 31/573; A61K 31/555; A61K 31/496; A61P 17/06; A61Q 5/00; A61K 8/63; A61K 8/23; A61K 8/58; A61K 8/49

(52) U.S. Cl. CPC A61K 33/04; A61K 31/573; A61K 31/555; A61K 31/496; A61K 8/4973; A61Q 5/006; A61K 8/63; A61K 8/23; A61K 8/58; A61P 17/06

(57) ABSTRACT

The present disclosure relates to treatments for psoriasis, seborrheic dermatitis, eczematous dermatitis and other related skin conditions. The treatment may comprise the topical application of a composition including a topical steroid, an antifungal agent, a keratolytic agent and a carrier. The composition may be included in a shampoo, an ointment, cream, lotion, paste, gel, solution, spray, aerosol, or oil appropriate for topical application. The various compositions may be in the form of a kit.

### CROSS-REFERENCE TO RELATED APPLICATION(S)

This application claims the benefit under 35 U.S.C. § 119(e) to U.S. Provisional Application 62/660,513, filed Apr. 20, 2018, and entitled “Compositions, Kits And Methods For The Treatment Of Psoriasis, Seborrheic Dermatitis, And Eczema Of The Head And Neck,” which is hereby incorporated herein by reference in its entirety for all purposes.

### FIELD OF THE INVENTION

The present disclosure relates to treatments for psoriasis, seborrheic dermatitis, eczematous dermatitis and other related skin conditions. More particularly the present disclosure relates to topical compositions for treatment of the above conditions.

### BACKGROUND

Severe psoriasis vulgaris affects approximately up to 10% of the population in the United States. In some instances, the condition is considered mild and described as seborrheic dermatitis or eczematous dermatitis. In other patients, the condition is severe and is classified as psoriasis vulgaris. Topical active product ingredients (APIs) are typically used as a first-line treatment for these chronic skin conditions. In some instances, an over-the-counter product is enough to improve the condition. In other individuals, prescription therapy is required as either a single agent or multiple agent treatment regimen. A classically described combination for psoriasis vulgaris includes a topical corticosteroid and vitamin D analogue. Each API is applied twice daily. One API is applied in the in the morning and in the evening, while the other API is applied before lunch and before dinner.

A complex skin application regimen such as the above example—requiring four different applications a day—leads to poor patient compliance and poor outcomes. At least one study suggests that reducing the frequency to once daily topical therapy increases patient compliance from 44% to 82% for patients with chronic skin diseases, such as psoriasis.

Patients with poor control of chronic skin conditions, such as psoriasis vulgaris, experience higher rates of depression, diabetes, heart disease, and lower quality of life. Brezinski E A, et al. Economic Burden of Psoriasis in the United States: A Systematic Review. JAMA Dermatol. 2015 June; 151(6):651-8. As such, there is a need in the art for improved methods of treating chronic skin conditions such as psoriasis vulgaris, seborrheic dermatitis, and eczematous dermatitis.

### BRIEF SUMMARY

Described herein are various implementations relating to compositions of and methods for treating psoriasis, seborrheic dermatitis, and eczema of the head and neck. In certain aspects, disclosed is a topical composition for the treatment of these conditions comprising a corticosteroid, an antifungal agent, keratolytic agent, and a carrier.

Disclosed herein is a kit for the treatment of psoriasis, seborrheic dermatitis, and/or eczema of the head and neck comprising a component comprising a therapeutically effective amount of a corticosteroid; a component comprising a therapeutically effective amount of a keratolytic agent; a component comprising a therapeutically effective amount of an antifungal agent and instructions for mixing and/or applying the forgoing components of to an affected area. In certain aspects, each of the foregoing components may be in the form of a shampoo, a cream, a lotion, a gel, a spray, a foam, a solution, or an ointment.

According to certain aspects, the corticosteroid is chosen from at least one of: hydrocortisone, clobetasol, fluocinolone, betamethasone, halobetasol, fluocinonide, and triamcinolone. In further aspects, the keratolytic agent is chosen from at least one of: salicylic acid, selenium sulfide, coal tar, and pyrithione zinc. In still further aspects, the antifungal agent is chosen from at least one of selenium sulfide, imidazoles, triazoles, allylamines, polyenes, and echinocandins selected from terbinafine, ciclopirox, econazole, ketoconazole, and nystatin.

According to certain embodiments, the corticosteroid is present at an amount from about 0.005% to about 2.5%. In further embodiments, the keratolytic agent is present at an amount from about 0.5% to about 40%. In yet further embodiments, the antifungal agent is present in an amount from about 0.5% to about 2%. According to still further embodiments, the corticosteroid is present in amount of about 1%, the keratolytic agent is present in an amount of about 2%, and the antifungal agent is present at an amount of about 1%.

Further disclosed herein is a kit for the treatment of psoriasis, seborrheic dermatitis, and/or eczema of the head and neck comprising: a hydrocortisone component; a zinc pyrithione component; c) a selenium disulfide component; and d) instructions for use thereof.

According to certain aspects, the hydrocortisone component is a foam, the zinc pyrithione component is a shampoo, and the selenium disulfide component is a shampoo. In further aspects, the hydrocortisone component comprises hydrocortisone in an amount from about 0.005% to about 2.5%, the zinc pyrithione component comprises zinc pyrithione at an amount from about 0.5% to about 40% and the selenium disulfide component comprises selenium disulfide in an amount from about 0.5% to about 2%. In further aspects, the hydrocortisone component comprises hydrocortisone in an amount of about 1%, the zinc pyrithione component comprises zinc pyrithione at an amount of about 2% and the selenium disulfide component comprises selenium disulfide in an amount of about 1%.

Further disclosed herein is a method of treating or preventing psoriasis, seborrheic dermatitis, and/or eczema of the head and neck of a subject in need thereof, the method comprising topically administering to at least one affected dermal area of the subject topical compositions comprising: a corticosteroid; a keratolytic agent; and antifungal agent. In certain aspects, the corticosteroid is a foam, the keratolytic agent is a shampoo, and the antifungal agent is a shampoo. In further aspects, the a keratolytic agent and the antifungal agent are applied on alternating days. In still further aspects, the corticosteroid is applied intermittently as needed.

In certain embodiments, the corticosteroid is in an amount from about 0.005% to about 2.5%, the keratolytic agent is in an amount from about 0.5% to about 40% and the antifungal agent is in an amount from about 0.5% to about 2%. In further embodiments, the corticosteroid is present in an amount of about 1%, the keratolytic agent is present in an amount of about 2%, and the antifungal agent is present in an amount of about 1%. In yet further embodiments, the corticosteroid is hydrocortisone, the keratolytic agent is zinc pyrithione, and the antifungal agent is selenium disulfide.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows exemplary instructions for application of the disclosed compositions, according to certain embodiments.

FIG. 2 is a chart of Dermatology Quality of Life Index score before and after seven days of treatment, according to certain embodiments, according to certain embodiments.

FIG. 3A shows a patient before treatment, according to certain embodiments.

FIG. 3B shows a patient after seven days of treatment, according to certain embodiments.

FIG. 3C shows a patient before treatment, according to certain embodiments.

FIG. 3D shows a patient after seven days of treatment, according to certain embodiments.

### DETAILED DESCRIPTION

It is understood that the implementations disclosed herein are not limited to specific synthetic methods or to particular reagents unless otherwise specified. It is also understood that the terminology used herein is for the purpose of describing particular aspects only and is not intended to be limiting. Although any methods and materials similar or equivalent to those described herein can be used in practicing the present invention, example methods and materials are now described for illustrative purposes.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it is understood that the particular value forms a further aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References to parts by weight of a particular element or component in a composition denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the term “subject” refers to the target of administration. Thus the subject disclosed methods can be a human, non-human primate, horse, pig, rabbit, dog, sheep, goat, cow, cat, guinea pig or rodent. The term does not denote a particular age or sex. Thus, adult and newborn subjects, whether male or female, are covered. In one aspect, the subject is a mammal. A patient refers to a subject afflicted with a disease or disorder.

The term “patient” includes human and veterinary subjects. In some aspects of the disclosed methods, the subject and/or patient has been diagnosed with a need for treatment of psoriasis vulgaris, seborrheic dermatitis, or eczema of the head and neck prior to the administering step.

As used herein, the term “treatment” refers to the medical management of a patient with the intent to cure, ameliorate, stabilize, or prevent a disease, pathological condition, or disorder. Treatment includes active treatment, that is, treatment directed specifically toward the improvement of a disease, pathological condition, or disorder, and also includes causal treatment, that is, treatment directed toward removal of the cause of the associated disease, pathological condition, or disorder. Treatment may also include palliative treatment, that is, treatment designed for the relief of symptoms rather than the curing of the disease, pathological condition, or disorder. Additionally, “treatment” includes preventative treatment, that is, treatment directed to minimizing or partially or completely inhibiting the development of the associated disease, pathological condition, or disorder. Treatment may also be supportive treatment, that is, treatment employed to supplement another specific therapy directed toward the improvement of the associated disease, pathological condition, or disorder. In various aspects, “treatment” includes any treatment of a subject, including a mammal, such as a human, and includes: (i) preventing the disease from occurring in a subject that can be predisposed to the disease but has not yet been diagnosed as having it; (ii) inhibiting the disease or arresting its development; or (iii) relieving the disease or causing regression of the disease.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with psoriasis vulgaris” means having been subjected to a physical examination by a person of skill, for example, a physician, and found to have a condition that can be diagnosed or treated by a compound or composition that can reduce the symptoms of psoriasis vulgaris. As a further example, “diagnosed with psoriasis” or “diagnosed with seborrheic dermatitis” or “diagnosed with eczema of the head and neck” refers to having been subjected to a physical examination by a person of skill, for example, a physician, and found to have a condition characterized by inflammation, hyperproliferation, cracking, scaling, and redness.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to psoriasis) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration. Such a diagnosis can be in reference to a disorder, such as psoriasis, and the like, as discussed herein.

As used herein, the terms “administering” and “administration” refer to any method of providing a pharmaceutical preparation to a subject. In certain embodiments, administering the composition involves applying a topical composition to an affected epidermal area of a subject suffering from psoriasis, seborrheic dermatitis, or eczema of the head and neck. As used herein, “affected epidermal area” refers to those patches of skin that exhibit common indicators of psoriasis, seborrheic dermatitis, or eczema of the head and neck including inflammation, hyperproliferation, cracking, scaling, and redness.

The term “topically administrable composition,” a “topical composition,” or a “topical formulation,” as used herein, means any formulation or composition which is pharmaceutically and/or cosmetically acceptable for topical delivery of the specified compounds according to embodiments of the invention.

Formulations suitable for topical application to the skin may take the form of an ointment, cream, lotion, paste, gel, solution, spray, aerosol, or oil. Carriers and excipients may be used. Carriers and excipients include, but are not limited to, Vaseline™, lanoline, polyethylene glycols, alcohols, and combinations thereof. The choice of topically administrable composition will depend on several factors, including the nature of the symptoms to be treated or prevented, the physiochemical characteristics of the particular compound to be administered and of other excipients present, their stability in the formulation, available manufacturing equipment, and cost constraints.

Disclosed herein is a topical composition for the treatment of psoriasis vulgaris, seborrheic dermatitis, or eczema of the head and neck including a corticosteroid, an antifungal agent, keratolytic agent, and a carrier. According to certain aspects, the topical corticosteroid is applied topically and a wide spectrum of different compounds may be used including at least one of hydrocortisone, clobetasol, fluocinolone, betamethasone, halobetasol, fluocinonide, triamcinolone, and other similar compounds. In certain embodiments, the topical corticosteroid is a mixture of the forgoing. In further aspects, the antifungal agent may be at least one of selenium sulfide, imidazoles, triazoles, allylamines, polyenes, and echinocandins such as terbinafine, ciclopirox, econazole, ketoconazole, and nystatin. In yet further aspects, the keratolytic may include at least one of salicylic acid, selenium sulfide, coal tar, and pyrithione zinc.

According to certain exemplary embodiments, the corticosteroid is hydrocortisone, the antifungal agent is selenium disulfide, and the keratolytic agent is pyrithione zinc.

In certain alternative embodiments, the corticosteroid is clobetasol; the antifungal agent ketoconazole; and the keratolytic agent is pyrithione zinc.

In some implementations, the corticosteroid is present at an amount from about 0.005% to about 2.5% w/w. In these and other implementations, the antifungal agent is present in an amount from about 0.5% to about 2% w/w. In various of these implementations, the keratolytic agent is present at an amount from about 0.5% to about 40% w/w.

In certain embodiments, hydrocortisone is present at an amount of about 1%; selenium disulfide is present in an amount of about 1%; and pyrithione zinc is present at an a amount of about 2%.

In certain alternative embodiments, Clobetasol is present at an amount of about 0.05%; ketoconazole is present in an amount of about 2%; and pyrithione zinc is present at an a amount of about 0.25%.

In various implementations, the composition is included in a shampoo, a cream, a lotion, a gel, a spray, a foam, a solution, or an ointment. In some implementations, the composition is in a shampoo. In some implementations, the shampoo contains selenium disulfide and pyrithione zinc and a foam contains hydrocortisone. In various of these implementations, the shampoo and/or foam may contain one or more inactive ingredients to stabilize and/or preserve the composition. Inactive ingredients, when present, may include at least one of chloro-m-cresol, citric acid, disodium edetate, ethoxylated alcohol, glycerin, 1,2,6-hexanetriol, methylparaben, parabens, potassium, sorbate, propyl gallate, propylene glycol, propyl paraben, sodium bisulfate, sodium citrate, butyl paraben, sodium metabisulfite, sorbic acid, tannic acid, zinc stearate, butylated hydroxytoluene, butylated hydroxyanisole, benzoic acid, salicylic acid, propyl paraben, dichlorobenzyl alcohol, formaldehyde, alpha-tocopherol, sodium ascorbate, ascorbic acid, ascorbyl palmitate phenol, m-cresol, bisphenol, cetrimide, benzalkonium chloride, sorbic acid, polyquatemum-1, chlorobutanol, chlorhexidine, Dowcell 200 (from Dow Chemical Co., Midland, Mich.), Glydant (dimethylol-25,5-dimethylhydantoin)(from Lonza, Inc. Fairlawn, N.J.), Germal 115 (imidazolidylurea) (from Sutton Laboratories, Chatham, N.J.), Germal II (diazolidinylurea) (from Sutton Laboratories, Chatham, N.J.), sodium hydroxymethylglycinate, Buzan 1504 (dimethhydroxymethylpyrazole) (from Buckman Labs, Memphis, Tenn.), phenoxyethanol, and benzoyl peroxide.

In various implementations, preservatives are present. A preservative, when present, may include at least one of hydroxylethyl benzene, hydroxylmethyl benzene, phenoxyethanol, propyl paraben, and methyl paraben, or other composition as would be recognized by those of skill in the art. Preservatives may comprise 0.1 to 10.00% by weight of the composition, and preferably 0.5 to 5.00% w/w.

The compositions may include other ingredients such as acid buffers. Acid buffers may include, but are not limited to, buffered solutions of acetic acid, formic acid, phosphoric acid, boric acid, citric acid, and ascorbic acid. Various implementations may include hydrophilicity modulators, such as polymers of 2-acrylamido-2-methylpropanesulfonic acid, alkyl sulfates, aryl sulfates, alkyl sulfonates, aryl sulfonates, or other compositions as would be appreciated by those of skill in the art. According to certain implementations, other ingredients may include keratin, collagen, amino acids, lecithin, aloe extracts, dimethicone, disodium EDTA, or other ingredients as desired and as would be appreciated by those of skill in the art.

Further disclosed herein is a kit for the treatment of psoriasis, seborrheic dermatitis, and eczema of the head and neck which may include a foam with a therapeutically effective amount of a topical steroid, a shampoo with a therapeutically effective amount of a keratolytic, a shampoo with a therapeutically effective amount of an antifungal agent and instructions for mixing and/or applying the components to an affected dermal area of a subject. In certain implementations, the disclosed kit comprises a foam having a therapeutically effective amount of hydrocortisone, a shampoo having a therapeutically effective amount of pyrithione zinc, and a shampoo having a therapeutically effective amount of selenium sulfide. In various implementations, hydrocortisone is present in amount of about 1%, pyrithione zinc is present in an amount of about 2%, and selenium sulfide is present in an amount of about 1%.

In various implementations, a kit may also comprise additional compounds and/or products co-packaged, co-formulated, and/or co-delivered with other components. In one example, an online distributor, drug store, supermarket, drug manufacturer, a drug reseller, a physician, a compounding shop, or a pharmacist can provide a kit comprising a disclosed compound(s) and/or product(s) and another component or components for delivery to a patient. It is contemplated that the disclosed kits can be used in connection with the disclosed methods of making and/or using the disclosed compositions.

In some implementations the kit may also comprise directions for use and/or other instructions. Various implementations of the directions may include a treatment regime and schedule. The directions may include how to apply the compositions, when to apply the compositions, the amount of composition the apply, the order in which to apply various compositions, and/or other directions as would be appreciated by those of skill in the art.

Further disclosed herein is a method of treating or preventing psoriasis, seborrheic dermatitis, and eczema of the head and neck in a subject in need thereof, the method comprising topically administering to an affected dermal areas of the subject a topical composition comprising a therapeutically effective amount of a corticosteroid, an antifungal agent, and keratolytic agent. In certain implementations, the corticosteroid is present at an amount between about 0.005% and about 0.5%, the antifungal agent is present in an amount between about 0.5% and about 2%, and the keratolytic agent is present at an amount between about 0.5% and about 40%.

In certain implementations the a keratolytic agent and the antifungal agent are applied on alternating days. In still further embodiments, the corticosteroid is applied intermittently as needed according to the level of severity of the symptoms.

According to certain exemplary embodiments of the disclosed method, the corticosteroid includes at least one of hydrocortisone, clobetasol, fuocinolone, betamethasone, halobetasol, fluocinonide, triamcinolone, or other corticosteriod as would be appreciated by those of skill in the art. In further aspects, the antifungal agent is at least one of terbinafine, ciclopirox, econazole, ketoconazole, terbinafine hydrochloride, butenafine hydrochloride, clotrimazole, miconazole nitrate, tolnaftate, or other antifungal agent as would be appreciated by those of skill in the art. In still further aspects, the keratolytic is at least one of salicylic acid, selenium sulfide, coal tar, pyrithione zinc, or other keratolytic agent as would be appreciated by one of skill in the art.

According to one implementation, the corticosteroid is clobetasol, the antifungal agent ketoconazole, and the keratolytic agent is pyrithione zinc. In these and other implementations, the clobetasol is present at an amount of about 0.05%, the ketoconazole is present in an amount of about 2%, and the pyrithione zinc is present at an a amount of about 0.25%.

In a further aspect, the topical composition is administered in an effective amount. In a yet further aspect, the effective amount is a therapeutically effective amount. In a still further aspect, the effective amount is a prophylactically effective amount. In a still further aspect, the method further comprises the step of identifying the mammal in need of treatment for psoriasis, seborrheic dermatitis, and/or eczema of the head and neck. In a yet further aspect, the method further comprises the step of identifying the mammal in need of prevention of psoriasis, seborrheic dermatitis, or eczema of the head and neck. In an even further aspect, the mammal has been diagnosed with a need for treatment of psoriasis, seborrheic dermatitis, and/or eczema of the head and neck prior to the administering step.

Without wishing to be bound by theory, it is hypothesized that corticosteroid reduces Th1 and Th17 lymphocyte infiltration, subsequent release of cytokines, reduces cell turnover, and inflammation in a number of dermatosis. A topical antifungal has multiple properties including a strong anti-inflammatory activity and a reduction of common skin-flora such as Malassezia Furur and Malassezia Globosa which are implicated in multiple chronic skin conditions including psoriasis vulgaris or seborrheic dermatitis. Hyperkeratosis, which is present in affected skin of psoriasis, seborrheic dermatitis, and eczema of the head and neck patients, reduces the penetration of the medications to the epidermis and dermis where the medications need to be delivered. A topical keratolytic agent will likely improve the compositions penetration into the affected skin.

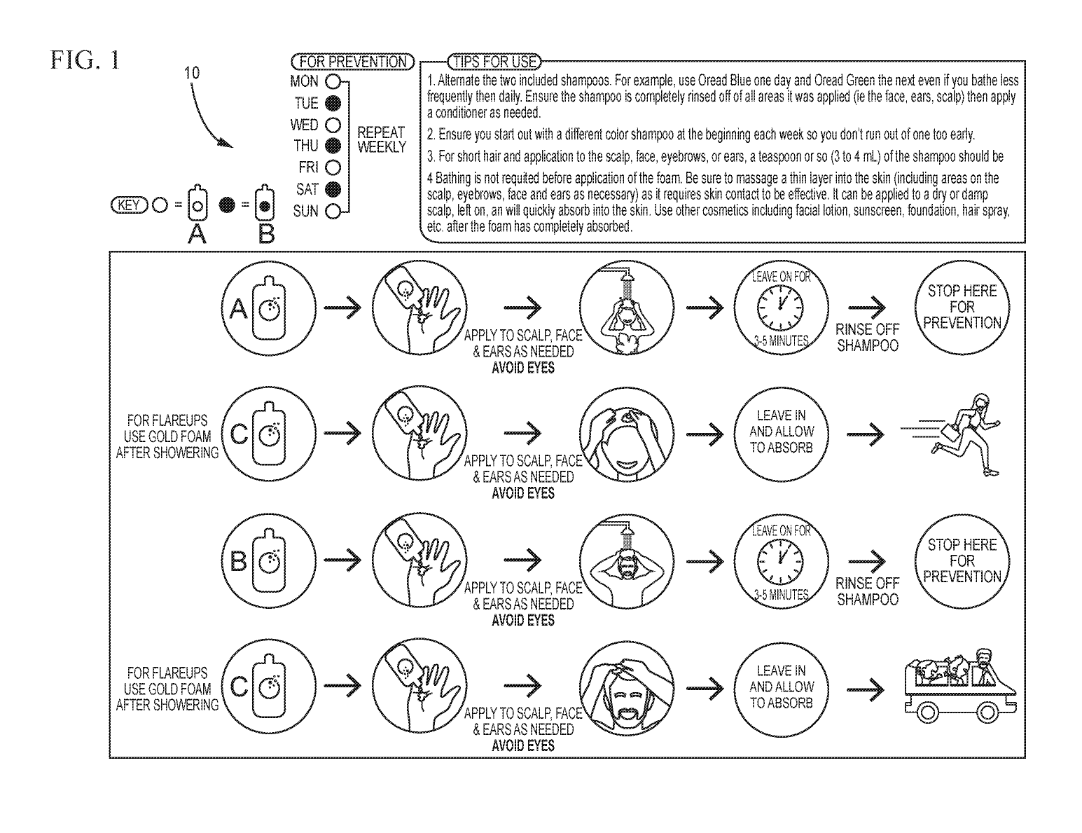

In various implementations, a kit is comprised of a zinc pyrithione shampoo, a selenium disulfide shampoo, a hydrocortisone foam and instructions for use thereof. FIG. 1 depicts exemplary instructions 10. The instructions 10 may include, among other items, a calendar depicting when to use each of the various compositions, instructions for how to apply and use the compositions, and instructions for how much of each composition to apply.

In the exemplary instructions of FIG. 1, a subject is to alternate the use of composition A and composition B each day. In this specific implementation composition A is the herein described selenium shampoo and composition B is the herein described pyrithione zinc shampoo. Both composition A and composition B are to be applied to the scalp, face, and ears of the subject as needed. The compositions are to be left on for about 3 to about 5 minutes and then rinsed.

In this specific implementation, composition C is a hydrocortisone composition, such as a foam. Composition C is to be applied to the scalp, face, and ears of the subject as needed. In this implementation, composition C is to be left in until absorbed by the skin.

In various implementation, about one teaspoon or about 3 to 4 mL of shampoo (compositions A or B) are to be applied. For certain subjects, especially those with long or thicker hair, a larger about of the composition may be applied.

In some implementation, the compositions should be applied regularly, such as every day or every other day. The compositions are applied to the skin and massaged into the skin. In some implementations the compositions may be applied to a dry scalp or a damp scalp.

### EXAMPLES

The following examples are illustrative and are not intended to limit the scope of this disclosure. Those of skill in the art will, in light of the present disclosure, appreciate that many changes can be made in the specific implementations disclosed herein and obtain a like or similar result without departing from the spirit and scope of the disclosure.

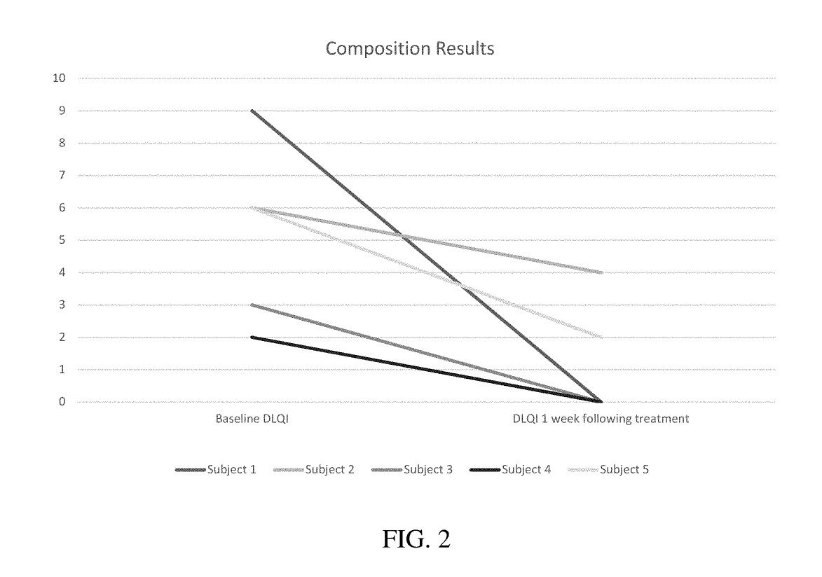

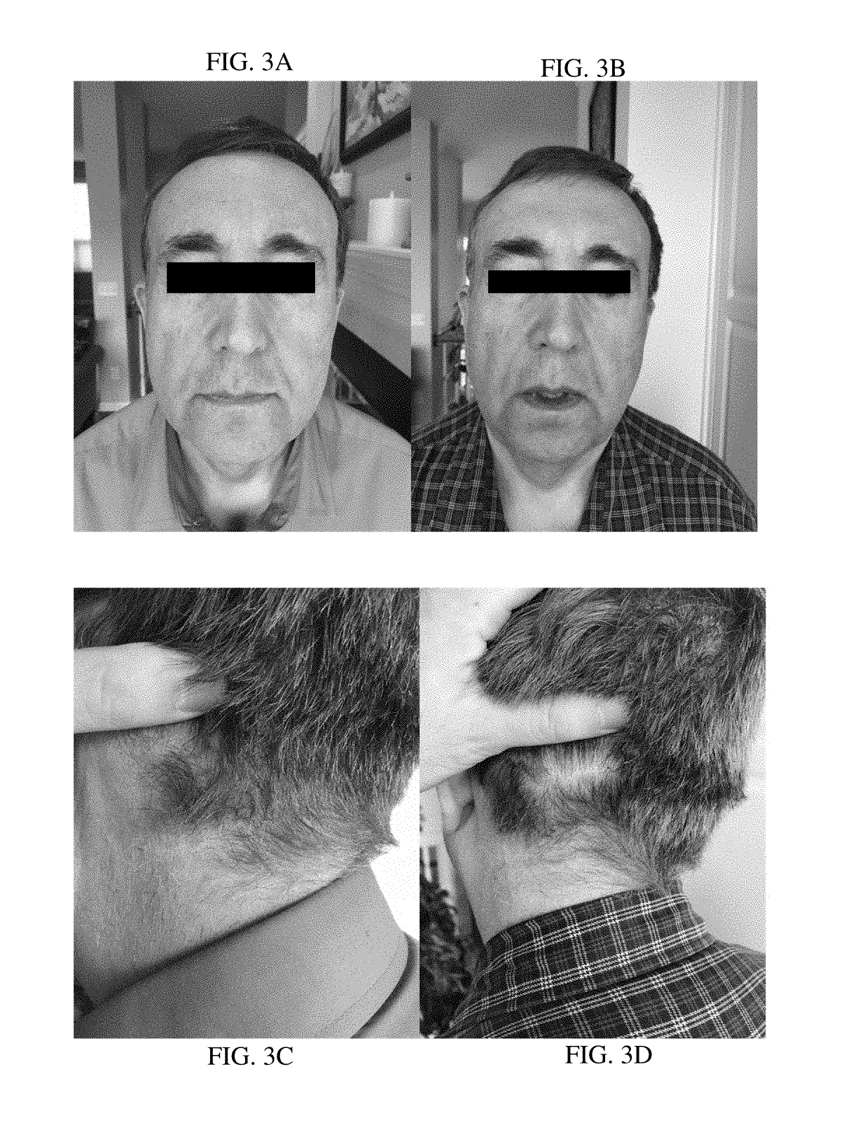

The topical application of the herein described compounds has an overall positive effective on the quality of life of patients with psoriasis, seborrheic dermatitis, eczema, and other related skin conditions, as shown in FIGS. 2 and 3A-D. Specifically, skin disease severity as measured by the Dermatology Quality of Life Index (“DLQI”). DLQI is a validated and frequently used tool to measure of skin disease severity in a number of skin conditions with high reliability. FIG. 2 is a graphical representation of the data of Table 1 and Table 2.

Before treatment subjects were assessed for a baseline DLQI. The subjects then were treated with the herein described treatment for one week and their DLQI was reassessed. Each subject reported statically significant a decrease in their DLQI after one week of treatment indicating a positive response to the treatment, with many showing complete resolution of their symptoms indicated by a total score of 0.

TABLE 1

| Subject | Baseline DLQI | DLQI 1 week following treatment |
|---|---|---|
| 1 | 9 | 0 |
| 2 | 6 | 4 |
| 3 | 3 | 0 |
| 4 | 2 | 0 |
| 5 | 6 | 2 |

TABLE 2

| | |
|---|---|
| Mean Baseline DLQI | 5.2 |
| Mean DLQI Following Treatment | 1.2 |
| T-test P value | 0.026693533 |

FIGS. 3A and 3B show the face of a subject before treatment and after one week of treatment. The figures demonstrate a decrease in red erythematous patches and scaling across the forehead, cheeks, nasal ala, glabella, upper lip, and chin in affected areas. FIGS. 3C and 3D show the occipital scalp and hairline of a subject before treatment and after one week of treatment. The patient experienced a significant decrease in symptoms represented in the photograph comparison such as redness, flaking, and patient-reported itching.

One specific example is a selenium shampoo formulation having the following composition:

| INCI Name | Trade Name | % W/W |
|---|---|---|
| Water | Deionized Water | 26.70 |
| Glycerin | Glycerin 99% Kosher | 1.50 |
| Methyl Gluceth-20 | Glucam E-20 Humectant | 2.50 |
| Disodium EDTA | Disodium EDTA | 0.10 |
| SELENIUM DISULFIDE | SELENIUM DISULFIDE USP | 1.00 |
| USP GRADE | GRADE | |
| PEG-12 Dimethicone | Emulsil S-393 | 3.00 |
| Ammonium Lauryl Sulfate | Colonial ALS | 24.30 |
| Ammonium Laureth Sulfate | Colonial ALES-2 | 13.90 |
| Cocamidopropyl Betaine | Colonial COAB | 11.50 |
| Cocamide MEA | Cola Mid CMA | 4.00 |
| Polyacrylate 33 | Rheomer 33T | 5.50 |
| Melaleuca Alternifolia | Lipovol Tea Tree | 1.00 |
| (Tea Tree) Leaf Oil | | |
| PEG-40 Hydrogenated | Hetoxide HC-40 | 4.00 |
| Castor Oil | | |
| Dehydroacetic Acid, | Geoguard 221 | 1.00 |
| Benzyl Alcohol, Water | | |
| | TOTAL: | 100.00 |

Another example is a hydrocortisone foam having the following composition:

| INCI Name | Trade Name | % W/W |
|---|---|---|
| Water | Deionized Water | 95.04 |
| Disodium EDTA | Disodium EDTA | 0.10 |
| Hydroxypropyl Methylcellulose | Tegocel HPM 4000 | 0.30 |
| Hydrocortisone | Hydrocortisone Base USP | 1.00 |
| Capryl/Capramidopropyl | Tego Betain 810 | 2.25 |
| Betaine | | |
| Citric Acid | Citric Acid 50% Stock | 0.31 |
| Dehydroacetic Acid, Benzyl | Geogard 221 | 1.00 |
| Alcohol | | |
| | | pH 4.5 |
| | TOTAL: | 100.00 |

Another example is a pyrithione zinc shampoo having the following composition:

| INCI Name | Trade Name | % W/W |
|---|---|---|
| Water | Deionized Water | 24.63 |
| Glycerin | Glycerin 99% Kosher | 1.50 |
| Phenoxyethanol, Piroctone Olamine | Nipaguard PO 5 | 1.00 |
| Zinc Pyrithione, Water | iDruff ZP | 4.17 |
| PEG-12 Dimethicone | Emulsil S-393 | 3.00 |
| Ammonium Lauryl Sulfate | Colonial ALS | 24.30 |
| Ammonium Laureth Sulfate | Colonial ALES-2 | 13.90 |
| Cocamidopropyl Betaine | Colonial COAB | 11.50 |
| Cocamide MEA | Cola Mid CMA | 4.00 |
| Polyacrylate 33 | Rheomer 33T | 4.50 |
| Methyl Gluceth-20 | Glucam E-20 Humectant | 2.50 |
| Melaleuca Alternifolia | Lipovol Tea Tree | 1.00 |
| (Tea Tree) Leaf Oil | | |
| PEG-40 Hydrogenated Castor Oil | Hetoxide HC-40 | 4.00 |
| | TOTAL: | 100.00 |

Another example is a topical composition having the following composition:

| | Water Solubility | Partition | Concentration |
|---|---|---|---|
| Component | (mg/L) | Coefficient | (% w/w) |
| Clobetasol | 3.86 | 3.50 | 0.05 |
| Ketoconazole | 0.9 | 4.35 | 2.00 |
| Pyrithione Zinc | 86.00 | 0.60 | 0.25 |
| Lotion/Shampoo | n/a | n/a | 97.70 |
| Base | | | |

Although the present disclosure has been described with references to various embodiments, persons skilled in the art will recognized that changes may be made in form and detail without departing from the spirit and scope of this disclosure.

What is claimed is:

1. A kit for the treatment of psoriasis, seborrheic dermatitis, and/or eczema of the head and neck comprising:
a) a component comprising a therapeutically effective amount of a corticosteroid;
b) a component comprising a therapeutically effective amount of a keratolytic agent;
c) a component comprising a therapeutically effective amount of an antifungal agent; and
d) instructions for mixing and/or applying the components of a)-c) to an affected area.

2. The kit of claim 1, wherein each of components of a)-c) are in the form selected from a list consisting of: a shampoo, a cream, a lotion, a gel, a spray, a foam, a solution, and an ointment.

3. The kit of claim 2, wherein the corticosteroid is chosen from at least one of: hydrocortisone, clobetasol, fluocinolone, betamethasone, halobetasol, fluocinonide, and triamcinolone.

4. The kit of claim 2, wherein the keratolytic agent is chosen from at least one of: salicylic acid, selenium sulfide, coal tar, and pyrithione zinc.

5. The kit of claim 2, wherein the antifungal agent is chosen from at least one of selenium sulfide, imidazoles, triazoles, allylamines, polyenes, and echinocandins selected from terbinafine, ciclopirox, econazole, ketoconazole, and nystatin.

6. The kit of claim 2, wherein the corticosteroid is present at an amount from about 0.005% to about 2.5%.

7. The kit of claim 6, wherein keratolytic agent is present at an amount from about 0.5% to about 40%.

8. The kit of claim 7, wherein the antifungal agent is present in an amount from about 0.5% to about 2%.

9. The kit of claim 8, wherein the corticosteroid is present in amount of about 1%, the keratolytic agent is present in an amount of about 2%, and the antifungal agent is present in an amount of about 1%.

10. A kit for the treatment of psoriasis, seborrheic dermatitis, and/or eczema of the head and neck comprising:
a) a hydrocortisone component;
b) a zinc pyrithione component;
c) a selenium disulfide component; and
d) instructions for use thereof.

11. The kit of claim 10, wherein the hydrocortisone component is a foam, the zinc pyrithione component is a shampoo, and the selenium disulfide component is a shampoo.

12. The kit of claim 11, wherein the hydrocortisone component comprises hydrocortisone in an amount from about 0.005% to about 2.5%, the zinc pyrithione component comprises zinc pyrithione at an amount from about 0.5% to about 40% and the selenium disulfide component comprises selenium disulfide in an amount from about 0.5% to about 2%.

13. The kit of claim 12, wherein the hydrocortisone component comprises hydrocortisone in an amount of about 1%, the zinc pyrithione component comprises zinc pyrithione at an amount of about 2%, and the selenium disulfide component comprises selenium disulfide in an amount of about 1%.

14. A method of treating or preventing psoriasis, seborrheic dermatitis, and/or eczema of the head and neck of a subject in need thereof, the method comprising topically administering to at least one affected dermal area of the subject topical compositions comprising:
a) a corticosteroid;
b) a keratolytic agent; and
c) an antifungal agent.

15. The method of claim 14, wherein the corticosteroid is a foam, the keratolytic agent is a shampoo, and the antifungal agent is a shampoo.

16. The method of claim 15, wherein the a keratolytic agent and the antifungal agent are applied in on alternating days.

17. The method of claim 16, wherein the corticosteroid is applied intermittently as needed.

18. The method of claim 16, wherein corticosteroid is in an amount from about 0.005% to about 2.5%, the keratolytic agent is in an amount from about 0.5% to about 40% and the antifungal agent is in an amount from about 0.5% to about 2%.

18. The method of claim 18, wherein the corticosteroid is present in amount of about 1%, the keratolytic agent is present in an amount of about 2%, and the antifungal agent is present at an amount of about 1%.

20. The method of claim 19, wherein the corticosteroid is hydrocortisone, the keratolytic agent is zinc pyrithione, and the antifungal agent is selenium disulfide.
